(19) United States
(12) Patent Application Publication
(10) Pub. No.: US 2024/0328888 A1
(43) Pub. Date: Oct. 3, 2024

(54) LEAK TESTING DEVICE

(71) Applicant: Solaire Medical Storage, LLC, Marne, MI (US)

(72) Inventors: Jeffrey Tibbe, Grand Haven, MI (US); Mark McClusky, Grand Rapids, MI (US)

(73) Assignee: Solaire Medical Storage, LLC, Marne, MI (US)

(21) Appl. No.: 18/621,293

(22) Filed: Mar. 29, 2024

Related U.S. Application Data
(60) Provisional application No. 63/493,557, filed on Mar. 31, 2023.

(51) Int. Cl. G01M 3/28; A61B 1/00

(52) U.S. Cl. CPC G01M 3/2853; A61B 1/00131; G01M 3/2815

(57) ABSTRACT

A leak testing device for performing a leak test on a medical device includes: an air compressor; an airflow assembly coupled to the compressor, defining an airflow passage, and configured to convey compressed air from the compressor to a medical device engaged with the airflow assembly; a pressure sensor in communication with compressed air within the airflow assembly and the medical device; a temperature sensor that senses water temperature within a water bath within which the medical device is submerged; an input device that receives an input indicating the time elapsed since the medical device was used in a medical procedure; and control circuitry that determines a pass or fail result of the leak test on the medical device based on data received from the pressure sensor, the temperature sensed by the temperature sensor, and the input received by the input device.

### CROSS-REFERENCE TO RELATED APPLICATION

This application claims the benefit of priority under 35 U.S.C. § 119 of U.S. Provisional Application No. 63/493,557, filed on Mar. 31, 2023, the entire contents of which is incorporated herein by reference.

### FIELD OF THE DISCLOSURE

The present disclosure generally relates to a leak testing device. More specifically, the present disclosure relates to a leak testing device for leak testing a medical device, such as an endoscope.

### BACKGROUND OF THE DISCLOSURE

Endoscopes are typically leak tested after use in a medical procedure by pressurizing then submerging the endoscopes in a water bath and manually checking for bubbles that indicate leakage. Leak testing devices that solely use pressure sensors with compressed air to leak test endoscopes are unreliable and often inaccurate. A leak testing device that reliably provides accurate leak testing results (pressure loss monitoring while the technician checks for bubbles) for an endoscope or other medical device that is submerged within water may be desired.

### SUMMARY OF THE DISCLOSURE

According to a first aspect of the present disclosure, a leak testing device for performing a leak test on a medical device includes: an air compressor configured to provide compressed air; an airflow assembly operably coupled to the air compressor, defining an airflow passage, and being configured to convey compressed air from the air compressor to a medical device selectively engaged with the airflow assembly; a pressure sensor in fluid communication with compressed air disposed within an interior volume that is defined by the airflow assembly and the medical device; a temperature sensor that senses a temperature of water within a water bath within which the medical device is submerged; an input device that receives an input indicating an amount of time elapsed since the medical device was used in a medical procedure; and control circuitry that determines a pass or fail result of the leak test on the medical device based on sensor data received from the pressure sensor, the temperature sensed by the temperature sensor, and the input received by the input device.

Embodiments of the first aspect of the disclosure can include any one or a combination of the following features:
the pressure sensor is a differential pressure sensor;
the differential pressure sensor senses a difference in pressure between the compressed air disposed within the interior volume that is defined by the airflow assembly and the medical device and compressed air that is disposed within a secondary interior volume defined by the airflow assembly, and the control circuitry determines the pass or fail result of the leak test based on the sensed difference in pressure;
a first valve operable between an open condition and a closed condition, wherein the first valve partitions the airflow passage into a proximal portion disposed between the first valve and the air compressor and a distal portion that is not in fluid communication with the air compressor, and a second valve operable between an open condition and a closed condition, wherein the second valve partitions the distal portion of the airflow passage into a first distal portion that is a portion of the interior volume and a second distal portion that is at least a portion of the secondary interior volume;
the proximal portion of the airflow passage is in fluid communication with the air compressor in the closed condition of the first valve;
at least one of a gauge pressure sensor and an absolute pressure sensor that is in fluid communication with the first distal portion of the airflow passage in the closed condition of the first valve and the closed condition of the second valve, the at least one of the gauge pressure sensor and the absolute sensor being configured to sense the pressure of compressed air in fluid communication with the at least one of the gauge pressure sensor and the absolute pressure sensor;
the control circuitry further determines the pass or fail result of the leak test based on the pressure sensed by the at least one of the gauge pressure sensor and the absolute pressure sensor;
the airflow assembly includes a manifold that defines the proximal portion, first distal portion, and second distal portion of the airflow passage, the manifold being a single unitary body;
the manifold defines a plurality of bore holes that form the proximal portion, first distal portion, and second distal portion of the airflow passage;
the control circuitry determines the pass or fail result of the leak test based on the sensed difference in pressure as a function of the sensed temperature;
the control circuitry determines the pass or fail result of the leak test based on the sensed difference in pressure as a function of time; and
the medical device is an endoscope.

According to a second aspect of the present disclosure, a method of performing a leak test on a medical device with a leak testing device includes the steps of pressurizing an interior volume that is at least partially defined by the medical device, receiving sensor data from a pressure sensor in fluid communication with the interior volume, sensing a temperature of water within a water bath in which the medical device is submerged, and determining a pass or fail result of the leak test based on the sensed temperature of the water and the sensor data received from the pressure sensor.

Embodiments of the second aspect of the disclosure can include any one or a combination of the following features:
the pressure sensor is a differential pressure sensor that senses a difference in pressure between compressed air within the interior volume and compressed air within a secondary interior volume that is not in fluid communication with the medical device;
the pass or fail result of the leak test is determined based on the sensed difference in pressure as a function of the sensed temperature;
the pass or fail result of the leak test is determined based on the sensed difference in pressure as a function of time;
the step of receiving, via an input device, an input indicating an amount of time elapsed since the medical device was used in a medical procedure, wherein the pass or fail result of the leak test is determined based on the received input;
the input device is a human-machine interface of the leak testing device; and
the human-machine interface comprises a touchscreen.

According to a third aspect of the present disclosure, a leak testing device for performing a leak test on a medical device includes: an air compressor configured to provide compressed air; an airflow assembly operably coupled to the air compressor and configured to convey compressed air from the air compressor to a medical device selectively engaged with the airflow assembly; a pressure sensor in fluid communication with compressed air disposed within an interior volume that is defined by at least a portion of the airflow assembly and the medical device; a temperature sensor that senses a temperature of water within a water bath within which the medical device is submerged; and control circuitry that determines a pass or fail result of the leak test on the medical device based on sensor data received from the pressure sensor and the temperature sensed by the temperature sensor.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a top perspective view of a leak testing device that is operably coupled with an endoscope that is submerged within water of a water bath, according to one embodiment;

FIG. 2 is a schematic diagram of an airflow assembly of a leak testing device, having a medical device operably coupled thereto, according to one embodiment;

FIG. 3 is a schematic diagram of an airflow assembly of a leak testing device that includes a plurality of gauge pressure sensors and a plurality of differential pressure sensors, according to one embodiment;

FIG. 4 is cross-sectional view of a manifold of a leak testing device that includes a plurality of bore holes that serve as airflow passages extending between various components of the leak testing device, including valves and pressure sensors, according to one embodiment;

FIG. 5 is a block diagram illustrating control circuitry of a leak testing device and various components of the leak testing device in communication with the control circuitry; and

FIG. 6 is a flow diagram of a method of leak testing a medical device with a leak testing device, according to one embodiment.

The components in the figures are not necessarily to scale, emphasis instead being placed upon illustrating the principles described herein.

### DETAILED DESCRIPTION

Additional features and advantages of the disclosure will be set forth in the detailed description which follows and will be apparent to those skilled in the art from the description, or recognized by practicing the disclosure as described in the following description, together with the claims and appended drawings.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

In this document, relational terms, such as “first” and “second,” “top” and “bottom,” and the like, are used solely to distinguish one entity or action from another entity or action, without necessarily requiring or implying any actual such relationship or order between such entities or actions.

For purposes of this disclosure, the term “coupled” (in all of its forms: couple, coupling, coupled, etc.) generally means the joining of two components (electrical or mechanical) directly or indirectly to one another. Such joining may be stationary in nature or movable in nature. Such joining may be achieved with the two components (electrical or mechanical) and/or any additional intermediate members. Such joining may include members being integrally formed as a single unitary body with one another (i.e., integrally coupled) or may refer to joining of two components. Such joining may be permanent in nature, or may be removable or releasable in nature, unless otherwise stated.

As used herein, the terms “the,” “a,” or “an,” mean “at least one,” and should not be limited to “only one” unless explicitly indicated to the contrary. Thus, for example, reference to “a component” includes embodiments having two or more such components unless the context clearly indicates otherwise.

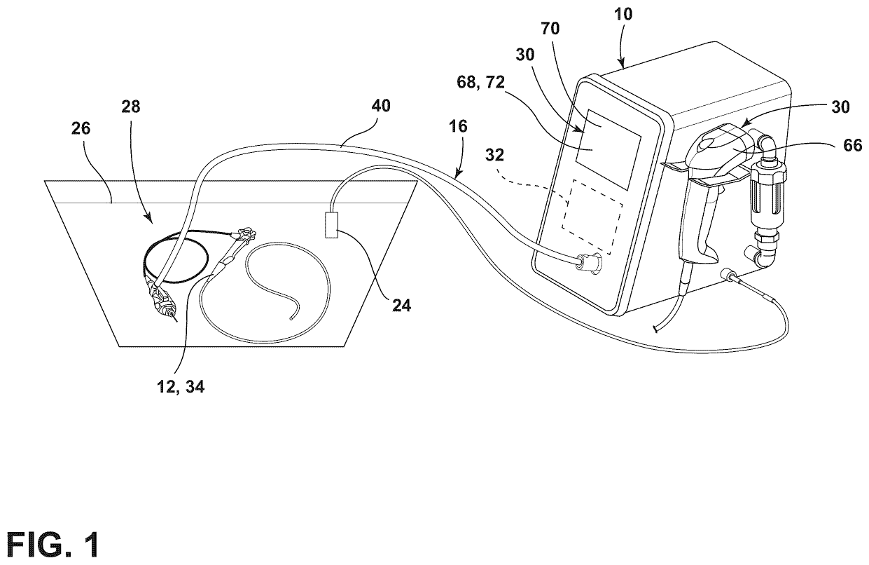

With reference to FIGS. 1-6, a leak testing device 10 for performing a leak test on a medical device 12 includes an air compressor 14 that is configured to provide compressed air. An airflow assembly 16 is operably coupled to the air compressor 14. The airflow assembly 16 defines an airflow passage 18 and is configured to convey compressed air from the air compressor 14 to a medical device 12 selectively engaged with the airflow assembly 16. A pressure sensor 20 is in fluid communication with compressed air disposed within an interior volume 22 that is defined by the airflow assembly 16 and the medical device 12. A temperature sensor 24 senses a temperature of water 26 within a water bath 28 within which the medical device 12 is submerged. The leak testing device 10 includes an input device 30 that receives an input indicating an amount of time elapsed since the medical device 12 was used in a medical procedure. The leak testing device 10 includes control circuitry 32 that determines a pass or fail result of the leak test on the medical device 12 based on sensor data received from the pressure sensor 20, the temperature sensed by the temperature sensor 24, and the input received by the input device 30.

Referring now to FIGS. 1-4, the leak testing device 10 is configured to perform a leak test on the medical device 12. In various embodiments, the medical device 12 is an endoscope 34, as illustrated in FIG. 1. A variety of types of medical devices 12 are contemplated, however. For example, the medical device 12 may be at least one of a host of types of medical devices 12 that include the interior volume 22, including, but not limited to, catheters, bags, scopes, and/or a variety of other medical instruments.

Referring now to FIGS. 1-4, the leak testing device 10 includes the airflow assembly 16. The medical device 12 is configured to be selectively engaged with the airflow assembly 16 for leak testing of the medical device 12. The air compressor 14 is operably coupled with the airflow assembly 16 to provide compressed air therein. The airflow assembly 16 is configured to convey the compressed air from the air compressor 14 to the medical device 12 that is selectively engaged with the airflow assembly 16. The airflow assembly 16 and the medical device 12 cooperate to define the interior volume 22 that is filled with compressed air during leak testing of the medical device 12, as described further herein.

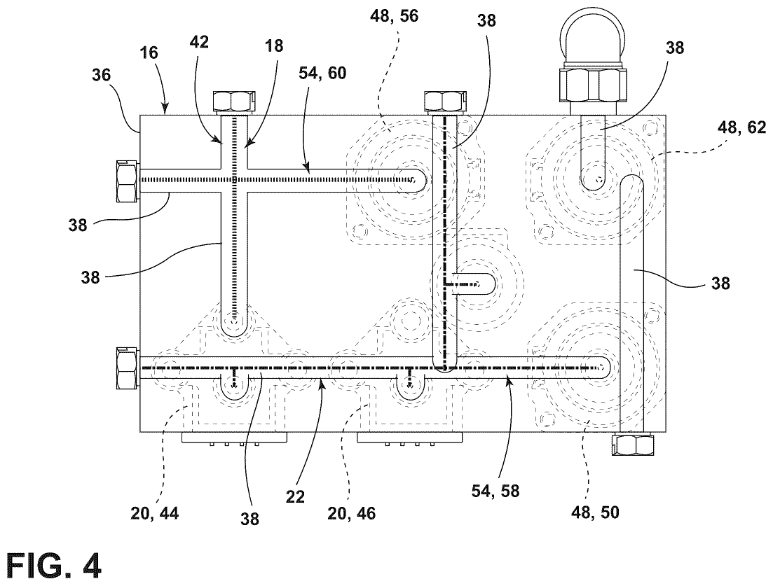

Referring still to FIGS. 1-4, the airflow assembly 16 defines the airflow passage 18. In various implementations, at least a portion of the airflow passage 18 defined by the airflow assembly 16 is a portion of the interior volume 22 defined by the airflow assembly 16 and the medical device 12. In some embodiments, the airflow assembly 16 includes a manifold 36 that defines the airflow passage 18. The manifold 36 can be a single unitary body with a plurality of holes 38 that cooperate to form the airflow passage 18. In various implementations, the manifold 36 is a single unitary body that includes a plurality of bore holes 38 (i.e., holes 38 bored out of the manifold 36) that form the airflow passage 18. The airflow passage 18 may be defined by components of the airflow assembly 16 in addition to the manifold 36, in various embodiments. For example, as illustrated in FIG. 4, bolts are fixed within certain bore holes 38 defined by the manifold 36 and serve to seal the openings of those bore holes 38 to confine the airflow passage 18. In some embodiments, the airflow assembly 16 can include a plurality of tubes connected by fittings. However, the manifold 36 may advantageously reduce the number of fittings relative to such systems and, therefore, reduce inaccuracies in a leak test of the medical device 12. As illustrated in FIG. 1, the airflow assembly 16 includes a conduit 40 that is configured to be selectively engaged with the medical device 12. The conduit 40 can extend between the manifold 36 and the medical device 12 and defines a portion of the interior volume 22 that is leak tested.

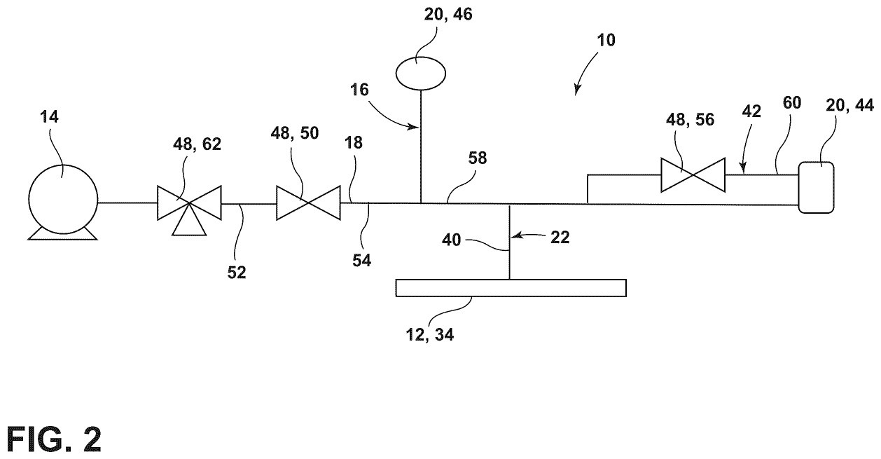

Referring now to FIGS. 2-5, the leak testing device 10 can include the pressure sensor 20. The pressure sensor 20 can be in fluid communication with compressed air disposed within the interior volume 22 that is defined by the airflow assembly 16 and the medical device 12. In some implementations, the pressure sensor 20 can be in fluid communication with compressed air disposed within a secondary interior volume 42 defined by the airflow assembly 16. In various embodiments, the secondary interior volume 42 defined by the airflow assembly 16 is isolated from (i.e., not in fluid communication with) the interior volume 22 that is defined by the airflow assembly 16 and the medical device 12, as described further herein. In some embodiments, the pressure sensor 20 may be in fluid communication with compressed air disposed within the interior volume 22 and with compressed air disposed within the secondary interior volume 42. For example, in an exemplary embodiment, the pressure sensor 20 of the leak testing device 10 is a differential pressure sensor 44 that is configured to sense a difference in pressure between compressed air in the interior volume 22 that is defined by the airflow assembly 16 and the medical device 12 and compressed air in the secondary interior volume 42 defined by the airflow assembly 16.

In some embodiments, the pressure sensor 20 of the leak testing device 10 can be a gauge pressure sensor 46 that senses the pressure of compressed air within a volume with respect to atmospheric pressure. In some implementations, the gauge pressure sensor 46 is in fluid communication with and configured to sense the pressure of compressed air within the interior volume 22 defined by the airflow assembly 16 and the medical device 12, as described further herein. In some embodiments, the pressure sensor 20 can be an absolute pressure sensor 20 that senses the pressure of the of compressed air within the interior volume 22 relative to absolute zero. It is to be understood that the absolute pressure sensor 20 and the gauge pressure sensor 46 may be utilized for generally the same purpose in various implementations of the leak testing device 10. As such, while embodiments of the leak testing device 10 described herein reference the gauge pressure sensor 46, specifically, it is contemplated that the absolute pressure sensor 20 may be utilized in such embodiments instead of or in addition to the gauge pressure sensor 46.

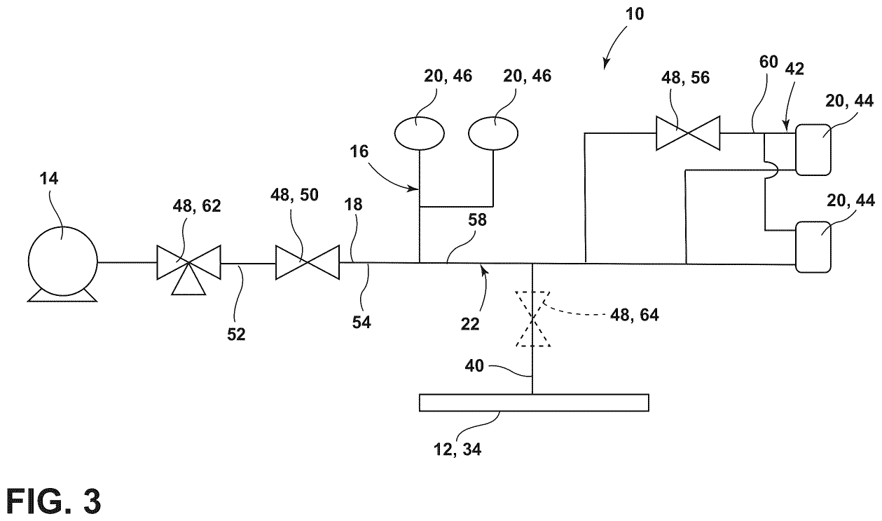

In various embodiments, the leak testing device 10 can include a plurality of pressure sensors 20. For example, in the embodiment illustrated in FIG. 2, the leak testing device 10 includes the gauge pressure sensor 46 that is configured to sense the pressure of compressed air within the airflow assembly 16, and the differential pressure sensor 44 that is in fluid communication with the interior volume 22 and the secondary interior volume 42 and configured to sense a difference in pressure thereof. In the embodiment illustrated in FIG. 3, the leak testing device 10 includes a plurality of differential pressure sensors 44, each of which is in fluid communication with both the interior volume 22 and the secondary interior volume 42, and a plurality of gauge pressure sensors 46 that are in fluid communication with at least a portion of the airflow passage 18. Having a plurality of differential pressure sensors 44 and/or a plurality of gauge pressure sensors 46 may advantageously increase accuracy or reliability (via redundancy) of the leak testing device 10. As illustrated in FIG. 4, the manifold 36 that defines the airflow passage 18 has a plurality of pressure sensors 20 coupled thereto, including the differential pressure sensor 44 and the gauge pressure sensors 46. In various implementations, the one or more pressure sensors 20 of the leak testing device 10 may be coupled with control circuitry 32 of the leak testing device 10 and configured to transmit sensor data, such as data pertaining to pressure of compressed air within an interior volume 22 and/or a difference in pressure between the interior volume 22 and the secondary interior volume 42, to the control circuitry 32, as described further herein.

Referring now to FIGS. 1-5, the leak testing device 10 can include a plurality of valves 48. In various implementations, the valves 48 may be configured to selectively partition various portions of the airflow passage 18. As illustrated exemplarily in FIGS. 2 and 3, in some embodiments, the leak testing device 10 includes a first valve 50 that is operable between an open condition and a closed condition. In the closed condition of the first valve 50, the valve 48 partitions the airflow passage 18 into a proximal portion 52 and a distal portion 54. As illustrated in FIG. 2, the proximal portion 52 is disposed between the first valve 50 and the air compressor 14. In various implementations, the proximal portion 52 of the airflow passage 18 is in fluid communication with the air compressor 14 in the closed condition of the first valve 50. The distal portion 54 of the airflow passage 18 is not in fluid communication with the air compressor 14 in the closed condition of the first valve 50. As such, closing the first valve 50 can isolate the distal portion 54 of the airflow passage 18 from the air compressor 14. In various implementations, one or more pressure sensors 20 of the leak testing device 10 are in fluid communication with the distal portion 54 of airflow passage 18. For example, as illustrated in FIG. 2, the differential pressure sensor 44 and the gauge pressure sensor 46 are in fluid communication with the distal portion 54 of the airflow passage 18.

As further illustrated in FIGS. 2 and 3, in various embodiments, the leak testing device 10 can include a second valve 56. The second valve 56 is operable between an open condition and a closed condition. In the closed condition, the second valve 56 partitions the distal portion 54 of the airflow passage 18 into a first distal portion 58 and a second distal portion 60. In various embodiments, the first distal portion 58 of the airflow passage 18 is a portion of the interior volume 22 that is defined cooperatively by the airflow assembly 16 and the medical device 12. As illustrated in FIG. 2, both the differential pressure sensor 44 and the gauge pressure sensor 46 are in fluid communication with the first distal portion 58 of the airflow passage 18. The second distal portion 60 of the airflow passage 18 is not in fluid communication with the interior volume 22 in the closed position of the second valve 56. In various implementations, the second distal portion 60 of the airflow passage 18 can be at least a portion of the secondary interior volume 42 that may be utilized in use of the leak testing device 10. For example, as illustrated in FIG. 2, the differential pressure sensor 44 is in fluid communication with the first distal portion 58 of the airflow passage 18 and is in fluid communication with the second distal portion 60 of the airflow passage 18 when the second valve 56 is in the closed position. As such, the differential pressure sensor 44 is in fluid communication with both the interior volume 22 defined by the airflow assembly 16 and the medical device 12 and the secondary interior volume 42 that is isolated from the interior volume 22 by the second valve 56 and defined by the airflow assembly 16. The differential pressure sensor 44 is, thus, operable to sense a difference in pressure between the interior volume 22 and the secondary interior volume 42 to determine a pass or fail result of a leak test on the medical device 12, as described further herein.

As illustrated in FIG. 2, the first distal portion 58 of the airflow passage 18 is disposed between the proximal portion 52 and the second distal portion 60. In the illustrated embodiment, the first distal portion 58 is in fluid communication with both the first valve 50 and the second valve 56 in the closed positions of the first and second valves 50, 56 during operation of the leak testing device 10. However, it is contemplated that additional valves 48 can be positioned between the first and second valves 50, 56, in some embodiments. In various embodiments, the leak testing device 10 can include one or more valves 48 in addition to the first valve 50 and the second valve 56. For example, as illustrated in FIGS. 2 and 3, the leak testing device 10 includes an exhaust valve 62 that is positioned within the proximal portion 52 of the airflow passage 18 between the air compressor 14 and the first valve 50. The illustrated exhaust valve 62 is a three-way valve that may function to exhaust compressed air from the leak testing device 10, as described further herein. In some embodiments, the leak testing device 10 may include a shut-off valve 64 positioned proximate to the conduit 40 to which the medical device 12 is configured to be selectively coupled. The shut-off valve 64 can be selectively closed to prevent compressed air from flowing into the conduit 40 in certain operating conditions of the leak testing device 10. Operation of the valves 48 of the leak testing device 10 can be controlled by control circuitry 32 of the leak testing device 10 that is coupled with the valves 48, as described further herein.

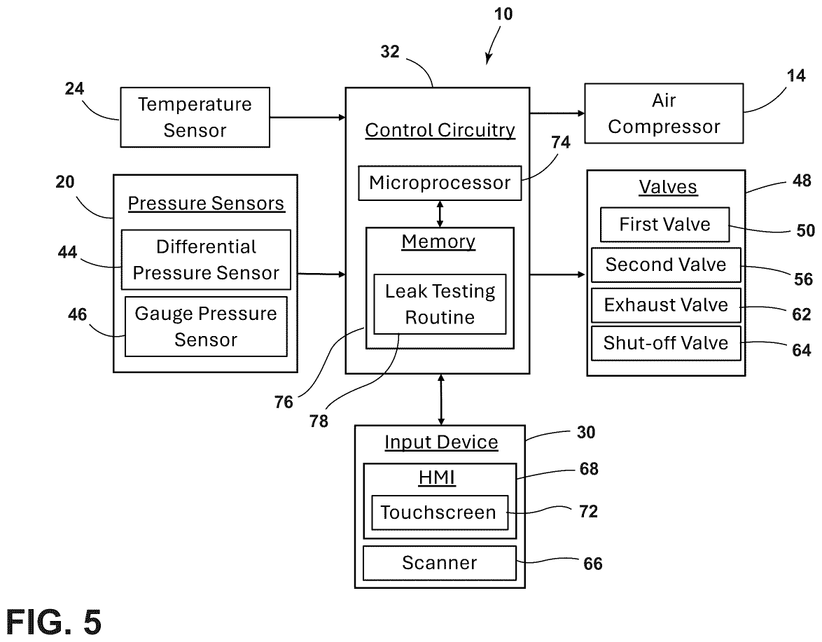

Referring now to FIGS. 1 and 5, the leak testing device 10 can include the temperature sensor 24. The temperature sensor 24 is configured to sense a temperature of water 26 within the water bath 28. As illustrated in FIG. 1, the temperature sensor 24 is positioned proximate a distal end of an elongated probe that extends outward from the leak testing device 10. The temperature sensor 24 is configured to sense the temperature of the water 26 within the water bath 28 and transmit corresponding temperature sensor data to control circuitry 32 of the leak testing device 10.

Referring now to FIGS. 1 and 5, the leak testing device 10 can include the input device 30 that is configured to receive an input. In various implementations, the input device 30 is configured to receive an input indicating an amount of time elapsed since the medical device 12 was used in a medical procedure. In some implementations, the input received by the input device 30 can indicate an amount of time the medical device 12 has been submerged within the water 26 of the water bath 28. In some implementations, the input device 30 is configured to recognize a code associated with the medical device 12 that is to be leak tested. For example, the input device 30 may be a scanner 66 for scanning a code or include a sensor for recognizing a code, such as a QR code or bar code. As illustrated in FIG. 1, the leak testing device 10 includes the scanner 66 that is operable to scan a code printed upon the medical device 12 and/or a label coupled to the medical device 12.

In some implementations, the input device 30 is a human-machine interface (“HMI”) 68 of the leak testing device 10. The HMI 68 may include a display 70, as illustrated in FIG. 1. The HMI 68 may further include the input device 30, which can be implemented by configuring the display 70 as a portion of a touchscreen 72 with circuitry to receive an input corresponding with a location over the display 70. Other forms of input devices 30, such as buttons and dials, can be used in place of or in addition to the touchscreen 72, in various implementations. In some implementations, the input device 30 may be a remote device, such as a handheld smart phone that is in wireless communication with the leak testing device 10. Various implementations are contemplated.

In various embodiments, the input device 30 receives the input that indicates an amount of time elapsed since the medical device 12 was used in a medical procedure. For example, in an exemplary embodiment wherein the input device 30 is the scanner 66, the input device 30 may scan a code associated with the medical device 12, to receive an input of information indicating when a previous medical procedure was completed, and thereby indicating an amount of time elapsed since the medical device 12 was used in a medical procedure. In another exemplary embodiment, wherein the input device 30 is the touchscreen 72, a user may utilize the touchscreen 72 to enter an input indicating a time elapsed since the medical device 12 was used in a medical procedure. Examples of types of data of an input received by the input device 30 that indicate a time elapsed since the medical device 12 was used in a medical procedure may include, but are not limited to, an end time of the medical procedure in which the medical device 12 was used, an end time of the use of the medical device 12 in the medical procedure, an amount of time since the end of the medical procedure in which the medical device 12 was used, or a time range within which the time elapsed since the medical device 12 was used in a medical procedure falls (e.g., 5-10 minutes).

Referring now to FIG. 5, the leak testing device 10 includes the control circuitry 32. The control circuitry 32 of the leak testing device 10 can be configured with a microprocessor 74 to process logic and routines stored in memory 76 that receives information from the above-described sensors and components, including the one or more pressure sensors 20, the valves 48, the temperature sensor 24, the one or more input devices 30, and/or the air compressor 14. The control circuitry 32 may generate information and commands as a function of all or a portion of the information received. Thereafter, the information and commands may be utilized to affect operation of the leak testing device 10 for the purpose of leak testing the medical device 12. The control circuitry 32 may include the microprocessor 74 and/or other analog and/or digital circuitry for processing one or more routines. Also, the control circuitry 32 may include the memory for storing one or more routines, such as a leak testing routine 78, described further herein.

It should be appreciated that the control circuitry 32 may include a stand-alone dedicated controller or may include a shared controller integrated with other control functions, such as those associated with the HMI 68. It should further be appreciated that one or more routines or subroutines of the leak testing device 10 may be carried out by a dedicated processor, in some implementations.

Referring now to FIGS. 1 and 5, in various implementations, the control circuitry 32 is configured to determine a pass or fail result of a leak test on the medical device 12 based on sensor data received from the pressure sensor 20, the temperature sensed by the temperature sensor 24, and the input received by the input device 30. In some implementations, wherein the leak testing device 10 includes the differential pressure sensor 44, the control circuitry 32 is configured to determine the pass or fail result of the leak test of the medical device 12 based on a difference in pressure between the interior volume 22 and the secondary interior volume 42 sensed by the differential pressure sensor 44. In some embodiments, the control circuitry 32 determines the pass or fail result of the leak test based on the sensed difference in pressure as a function of the sensed temperature. In some implementations, the control circuitry 32 determines the pass or fail result of the leak test based on the sensed difference in pressure between the interior volume 22 and the secondary interior volume 42 as a function of time.

In an exemplary embodiment, the control circuitry 32 is configured to determine the pass or fail result of the leak test based on the sensed difference in pressure between the interior volume 22 and the secondary interior volume 42 as a function of temperature and time. For example, the differential pressure sensor 44 may sense a difference in pressure between the interior volume 22 partially defined by the medical device 12 and the secondary interior volume 42 that is isolated from the interior volume 22, and the temperature sensor 24 may sense the temperature of water 26 within a water bath 28 in which the medical device 12 is submerged over a span of time. Based on the sensed pressure differential as a function of time and temperature over the span of time, leak rate values are determined and compared with corresponding threshold values to determine the pass or fail result of the leak test of the medical device 12. In various implementations, the threshold values with which the leak rate values are compared in the leak test are also determined as a function of the sensed temperature and/or time. As such, the threshold value that the determined leak rate value is compared to at a given point in time may be different from the threshold value at another point in time. In some embodiments, the control circuitry 32 1may determine that the medical device 12 fails the leak test based on the leak rate value reaching the corresponding threshold value at any point in time during the leak test of the medical device 12. This may advantageously account for leaks in, for example, an endoscope 34 that are only revealed briefly before artificially sealing once more during flexing of the endoscope 34 during the leak test of the endoscope 34.

It is contemplated that various types of sensor data from the pressure sensor 20 may be utilized by the control circuitry 32 to determine the pass or fail result of the leak test. For example, in some embodiments, the control circuitry 32 may utilize sensor data pertaining to pressure (e.g., received from the gauge pressure sensor 46) and/or sensor data pertaining to pressure differential (e.g., received from the differential pressure sensor 44). In some implementations, the input received by the input device 30 indicating the time elapsed since the medical device 12 was used in a medical procedure and/or the input indicating an amount of time the medical device 12 has been submerged within the water 26 of the water bath 28 is relayed to the control circuitry 32, and the control circuitry 32 determines the pass or fail result of the leak test based on, in part, the input.

Referring now to FIGS. 1-6, the leak testing device 10 is configured to execute the leak testing routine 78 for leak testing the medical device 12. In operation of the leak testing device 10 that utilizes an exemplary leak testing routine 78, initially, a user of the leak testing device 10 receives a medical device 12, here, an endoscope 34, that was previously used in a medical procedure. The leak testing routine 78 begins with the input device 30 of the leak testing device 10 receiving an input. In various implementations, a code is received by the input device 30. For example, a user badge barcode and/or a medical device barcode may be scanned by the scanner 66 or input into the HMI 68 by the user. These codes may determine a future workflow in the leak testing routine 78. For example, an endoscope 34 having a specific serial number may be restricted to wet testing, while other medical devices 12 may be permissibly dry tested for leaks. In some implementations, the input received by the scanner 66 may indicate a time elapsed since the endoscope 34 was used in a medical procedure. This information can be of critical importance to ensuring an accurate leak test, as the temperature of the endoscope 34 can impact the results of the leak test and is directly tied to the time elapsed since the endoscope 34 was used in a previous medical procedure, as the endoscope 34 is generally elevated to the temperature of the human body during the procedure and cools off gradually thereafter.

In some implementations, the input device 30 receives the input indicating the amount of time elapsed since the endoscope 34 was used in a medical procedure via use of the human machine interface 68 by the user. For example, the user may select a time range option present on the touchscreen 72 indicating the amount of time elapsed or enter the amount of time elapsed into the touchscreen 72. The input received by the input device 30 is communicated to the control circuitry 32 for use in determining the pass or fail result of the leak test.

Next, the user connects the endoscope 34 to the airflow assembly 16 via the conduit 40. The leak testing routine 78 continues with the leak testing device 10 entering a pre-testing phase. In the pre-testing phase, the control circuitry 32 prompts the air compressor 14 to provide compressed air within the airflow assembly 16 and the attached endoscope 34. The gauge pressure sensor 46 that is in fluid communication with the airflow assembly 16 senses the pressure within the interior volume 22 cooperatively defined by the airflow assembly 16 and the endoscope 34 to determine that the endoscope 34 is correctly connected to the conduit 40 of the airflow assembly 16 and/or to identify major leaks in the endoscope 34 before submersion of the endoscope 34 into the water bath 28. Running the air compressor 14 in the pre-test phase also has the effect of seating seals (e.g., O-rings) of the leak testing device 10 for optimal accuracy in the leak testing procedure. In order to sufficiently seat the seals, a desired pre-test pressure must be reached, which is sensed by the gauge pressure sensor 46.

In use of the system, endoscopes 34 are often slightly folded during this pre-test phase, which kinks the endoscope 34 and isolates a portion of the interior volume 22 of the endoscope 34. As such, while conducting the pre-test compressor run, pressure can build within the airflow assembly 16 and the interior volume 22 of the endoscope 34 upstream of the kink and reach the desired pressure for seating O-rings. Subsequently, the pressure of the compressed air against the endoscope 34 can overcome the kink to reveal the isolated portion of the interior volume 22 of the endoscope 34. Pressure rapidly decreases as a result, which may cause false indications of a major leak within the endoscope 34 or a faulty connection between the endoscope 34 and the conduit 40. Accordingly, in some implementations of the leak testing routine 78, the air compressor 14 may provide compressed air for a period of time beyond when the desired pre-test pressure within the airflow assembly 16 is reached, as sensed by the gauge pressure sensor 46. This allows kinks and folds within the endoscope 34 to be revealed prior to a false leak indication being triggered. In some embodiments, the control circuitry 32, gauge pressure sensor 46, and air compressor 14 may communicate, such that the initial amount of time that it takes for the pressure within the interior volume 22 defined by the airflow assembly 16 and the endoscope 34 to reach the desired initial pressure for seating O-rings is recorded. The control circuitry 32 may then control the air compressor 14 to continue providing compressed air into the interior volume 22 for a total time period that is equal to the initial recorded time period multiplied by a multiplier (e.g., 4×the initial time). As such, false indications of leaks may be avoided during the pre-test compressing stage.

After the pre-test phase when it is determined that the endoscope 34 is correctly connected to the airflow assembly 16 via the conduit 40 and that there are no major leaks in the airflow assembly 16, the leak testing routine 78 continues with the leak testing phase. In the leak testing phase, the air compressor 14 is run until a target leak testing pressure is reached as sensed by the gauge pressure sensor 46. At this point, the user submerges the endoscope 34 within the water 26 within the water bath 28. Once the endoscope 34 is submerged within the water 26, the user may allow for a short period of time to allow the endoscope 34 to acclimate to the water 26. Further, at this junction, or at a previous time, the temperature sensor 24 is positioned by the user within the water 26 of the water bath 28 such that the temperature sensor 24 is configured to sense the temperature of the water 26.

With the endoscope 34 and the temperature sensor 24 submerged within the water 26 of the water bath 28, the leak testing phase continues. Once the gauge pressure sensor 46 senses that the target pressure is reached, the control circuitry 32 controls the first valve 50, as illustrated in FIG. 2, to move from the open condition to the closed condition. This partitions the airflow passage 18 into the proximal portion 52 that is disposed between the first valve 50 and the air compressor 14 and the distal portion 54 that is not in fluid communication with the air compressor 14. In this way, the air compressor 14 is isolated from the distal portion 54 of the airflow passage 18 which is in fluid communication with the endoscope 34. Isolating the air compressor 14 from the endoscope 34 via the first valve 50 in this way may advantageously increase the accuracy of the leak test, as air compressors 14 are often inconsistent in operation, such that a leak test that is in fluid communication with the air compressor 14 may result in false leak detections due to the operation of the air compressor 14.

Next, the control circuitry 32 controls the second valve 56 to enter the closed condition from the open condition, which partitions the distal portion 54 of the airflow passage 18 into the first distal portion 58 and the second distal portion 60. The first distal portion 58 forms a portion of the interior volume 22 that is defined by the endoscope 34 and the airflow assembly 16, and the second distal portion 60 forms at least a portion of the secondary interior volume 42. The differential pressure sensor 44 is in fluid communication with the first and second distal portions 58, 60 of the airflow passage 18, and thereby is in fluid communication with the secondary interior volume 42 and the interior volume 22, such that the differential pressure sensor 44 is operable to sense a pressure differential between the interior volume 22 and the secondary interior volume 42.

With the first and second valves 50, 56 in the closed conditions, the temperature sensor 24 senses the temperature of the water 26, the differential pressure sensor 44 senses the pressure differential between the interior volume 22 and the secondary interior volume 42, and the gauge pressure sensor 46 senses the pressure within the interior volume 22 for a period of time. In various embodiments, the period of time is a predetermined period of time (e.g., 90 seconds). Sensor data from the temperature sensor 24, differential pressure sensor 44 and gauge pressure sensor 46 is transmitted to the control circuitry 32 for use in determining a pass or fail result of the leak test, as described further herein. During the aforementioned period of time, the user may manipulate the submerged endoscope 34 by adjusting knobs or flexing the tube portion, which may reveal leaks by uncovering artificially sealed perforations in the endoscope 34.

Subsequent to or concurrent with the period of time in which sensor data is collected and transmitted to the control circuitry 32 while the endoscope 34 is submerged, the leak testing routine 78 continues with the control circuitry 32 determining a pass or fail result of the leak test of the endoscope 34. In various implementations, the pass or fail result of the leak test of the endoscope 34 is determined by the control circuitry 32 based on the input from the input device 30 indicating the time elapsed since the endoscope 34 was used in a medical procedure, the temperature sensed by the temperature sensor 24, the pressure differential sensed by the differential pressure sensor 44, and/or the pressure sensed by the gauge pressure sensor 46, as described previously herein. The input device 30 may also receive an input from the user indicating that bubbles originating from the submerged endoscope 34 are detected, indicating a leak, which would result in a determination that the endoscope 34 failed the leak test.

In some implementations, wherein rapid pressure loss is detected and indicates a large leak from the endoscope 34, the control circuitry 32 may control the first valve 50 to re-enter the open condition and the air compressor 14 to provide compressed air to the airflow assembly 16 and the attached endoscope 34. Doing so may allow for the user to inspect the leak for an extended amount of time, as bubbles will continuously be emitted from the leak in the submerged endoscope 34 due to the continuous flow of compressed air into the endoscope 34. Further, continuously providing compressed air into the endoscope 34 while it is submerged within the water bath 28 may advantageously prevent water 26 from entering the interior volume 22 of the endoscope 34.

Next, the leak testing routine 78 enters a depressurization phase, wherein the control circuitry 32 controls the first and second valves 50, 56 to reset to the open conditions and operation of the air compressor 14 ceases if not already stopped. In various implementations, the user has already removed the endoscope 34 from the water bath 28 at the initiation of the depressurization phase. The control circuitry 32 may also be configured to control the exhaust valve 62 during the depressurization phase to exhaust compressed air from the airflow assembly 16. It is contemplated that, in some implementations, the leak testing device 10 may prompt a user to choose an optional retest function via the HMI 68. In such embodiments, the leak testing device 10 may be configured to account for acclimation that the endoscope 34 has undergone due to being submerged in the water bath 28 previously, if the retest option is chosen by the user. As such, the control circuitry 32 may determine the pass or fail result of the subsequent leak test based on the test being a re-test of the endoscope 34. It is to be understood that, although the execution of the leak testing routine 78 is described herein with reference to the endoscope 34, various types of medical devices 12 may be leak tested in this manner. Further, it is understood that the leak testing routine 78 described herein is exemplary in nature and various leak testing routines 78 executed by the leak testing device 10 may include more or fewer phases and/or steps than the exemplary leak testing routine 78 described herein.

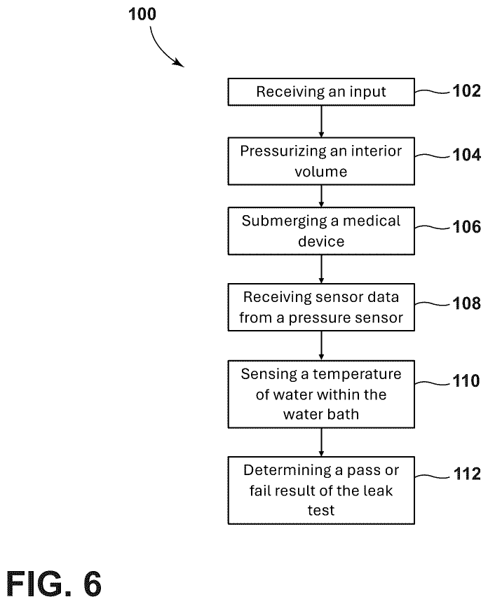

Referring now to FIG. 6, a method 100 of performing a leak test on the medical device 12 with the leak testing device 10 is illustrated. The method 100 may include a step 102 of receiving an input indicating an amount of time elapsed since the medical device 12 was used in a medical procedure. In various implementations, the input device 30 of the leak testing device 10 receives the input indicating the amount of time elapsed since the medical device 12 was used in the medical procedure. In some embodiments, the input device 30 may be the HMI 68 of the leak testing device 10. For example, as illustrated in FIG. 1, the leak testing device 10 includes the input device 30 in the form of the touchscreen 72 that forms at least a portion of the HMI 68 of the leak testing device 10. It is contemplated that the input device 30 may be the scanner 66 that is configured to receive the input in the form of a code as described previously herein.

The method 100 can include a step 104 of pressurizing the interior volume 22 that is at least partially defined by the medical device 12. In various implementations, the air compressor 14 of the leak testing device 10 is utilized to provide compressed air to the interior volume 22 that is defined at least partially by the medical device 12. In some implementations, the step 104 of pressurizing the interior volume 22 is performed by first providing compressed air to the interior volume 22 and then subsequently controlling the first valve 50 and the second valve 56 of the leak testing device 10 to partition the airflow passage 18 of the airflow assembly 16 into the proximal portion 52, the first distal portion 58, and the second distal portion 60. As such, the interior volume 22 that is formed, in part by the first distal portion 58 of the airflow passage 18 and defined in part by the medical device 12, is pressurized with compressed air that is isolated from the air compressor 14.

Referring still to FIG. 6, the method 100 can include a step 106 of submerging the medical device 12 within water 26 of the water bath 28. In various implementations, a user may manually submerge the endoscope 34 that is connected with the leak testing device 10 into the water bath 28. The method 100 may further include a step 108 of receiving sensory data from the pressure sensor 20 in fluid communication with the interior volume 22. In some implementations, the pressure sensor 20 is the differential pressure sensor 44 that senses a difference in pressure between compressed air within the interior volume 22 and compressed air within the secondary interior volume 42 that is not in fluid communication with the medical device 12. For example, as illustrated in FIG. 2, the differential pressure sensor 44 is in fluid communication with the interior volume 22 that is formed partially by the first distal portion 58 of the airflow passage 18, and the differential pressure sensor 44 is in fluid communication with the secondary interior volume 42 that is formed at least in part by the second distal portion 60 of the airflow passage 18.

Referring still to FIG. 6, the method 100 can include a step 110 of sensing a temperature of the water 26 within the water bath 28 in which the medical device 12 is submerged. As illustrated in FIG. 1, in various implementations, the temperature sensor 24 may be in contact with the water 26 within the water bath 28 in which the medical device 12 is submerged, such that the temperature sensor 24 is operable to sense the temperature of the water 26.

The method 100 may include a step 112 of determining a pass or fail result of the leak test. In some implementations, the pass or fail result of the leak test is determined by the control circuitry 32 of the leak testing device 10 based on the sensed temperature of the water 26, the sensor data received from the pressure sensor 20, and/or the input indicating an amount of time elapsed since the medical device 12 was used in the medical procedure received by the input device 30. In some implementations, the pass or fail result determined at step 112 is determined based on the sensed difference in pressure between the interior volume 22 and the secondary interior volume 42 as a function of temperature. In some implementations, the pass or fail result of the leak test determined at step 112 is determined based on the sensed difference in pressure between the interior volume 22 and the secondary interior volume 42 as a function of time. In some implementations, the pass or fail result of the leak test determined at step 112 is determined based on the received input indicating the amount of time elapsed since the medical device 12 was used in the medical procedure. In an exemplary embodiment, the pass or fail result of the leak test determined at step 112 is determined based on the input received, and the sensed difference in pressure between the interior volume 22 and the secondary interior volume 42 as a function of time and as a function of the temperature sensed by the temperature sensor 24.

What is claimed is:

1. A leak testing device for performing a leak test on a medical device, comprising:
an air compressor configured to provide compressed air;
an airflow assembly operably coupled to the air compressor, defining an airflow passage, and being configured to convey compressed air from the air compressor to a medical device selectively engaged with the airflow assembly;
a pressure sensor in fluid communication with compressed air disposed within an interior volume that is defined by the airflow assembly and the medical device;
a temperature sensor that senses a temperature of water within a water bath within which the medical device is submerged;
an input device that receives an input indicating an amount of time elapsed since the medical device was used in a medical procedure; and
control circuitry that determines a pass or fail result of the leak test on the medical device based on sensor data received from the pressure sensor, the temperature sensed by the temperature sensor, and the input received by the input device.

2. The leak testing device of claim 1, wherein the pressure sensor is a differential pressure sensor.

3. The leak testing device of claim 2, wherein the differential pressure sensor senses a difference in pressure between the compressed air disposed within the interior volume that is defined by the airflow assembly and the medical device and compressed air that is disposed within a secondary interior volume defined by the airflow assembly, and wherein the control circuitry determines the pass or fail result of the leak test based on the sensed difference in pressure.

4. The leak testing device of claim 3, further comprising:
a first valve operable between an open condition and a closed condition, wherein the first valve partitions the airflow passage into a proximal portion disposed between the first valve and the air compressor and a distal portion that is not in fluid communication with the air compressor; and
a second valve operable between an open condition and a closed condition, wherein the second valve partitions the distal portion of the airflow passage into a first distal portion that is a portion of the interior volume and a second distal portion that is at least a portion of the secondary interior volume.

5. The leak testing device of claim 4, wherein the proximal portion of the airflow passage is in fluid communication with the air compressor in the closed condition of the first valve.

6. The leak testing device of claim 4, further comprising:
at least one of a gauge pressure sensor and an absolute pressure sensor that is in fluid communication with the first distal portion of the airflow passage in the closed condition of the first valve and the closed condition of the second valve, the at least one of the gauge pressure sensor and the absolute sensor being configured to sense the pressure of compressed air in fluid communication with the at least one of the gauge pressure sensor and the absolute pressure sensor.

7. The leak testing device of claim 6, wherein the control circuitry further determines the pass or fail result of the leak test based on the pressure sensed by the at least one of the gauge pressure sensor and the absolute pressure sensor.

8. The leak testing device of claim 4, wherein the airflow assembly comprises:
a manifold that defines the proximal portion, first distal portion, and second distal portion of the airflow passage, the manifold being a single unitary body.

9. The leak testing device of claim 8, wherein the manifold defines a plurality of bore holes that form the proximal portion, first distal portion, and second distal portion of the airflow passage.

10. The leak testing device of claim 3, wherein the control circuitry determines the pass or fail result of the leak test based on the sensed difference in pressure as a function of the sensed temperature.

11. The leak testing device of claim 10, wherein the control circuitry determines the pass or fail result of the leak test based on the sensed difference in pressure as a function of time.

12. The leak testing device of claim 1, wherein the medical device is an endoscope.

13. A method of performing a leak test on a medical device with a leak testing device, comprising the steps of:
pressurizing an interior volume that is at least partially defined by the medical device;
receiving sensor data from a pressure sensor in fluid communication with the interior volume;
sensing a temperature of water within a water bath in which the medical device is submerged; and
determining a pass or fail result of the leak test based on the sensed temperature of the water and the sensor data received from the pressure sensor.

14. The method of claim 13, wherein the pressure sensor is a differential pressure sensor that senses a difference in pressure between compressed air within the interior volume and compressed air within a secondary interior volume that is not in fluid communication with the medical device.

15. The method of claim 14, wherein the pass or fail result of the leak test is determined based on the sensed difference in pressure as a function of the sensed temperature.

16. The method of claim 14, wherein the pass or fail result of the leak test is determined based on the sensed difference in pressure as a function of time.

17. The method of claim 13, further comprising the step of:
receiving, via an input device, an input indicating an amount of time elapsed since the medical device was used in a medical procedure, wherein the pass or fail result of the leak test is determined based on the received input.

18. The method of claim 17, wherein the input device is a human-machine interface of the leak testing device.

19. The method of claim 18, wherein the human-machine interface comprises a touchscreen.

20. A leak testing device for performing a leak test on a medical device, comprising:
an air compressor configured to provide compressed air;
an airflow assembly operably coupled to the air compressor and configured to convey compressed air from the air compressor to a medical device selectively engaged with the airflow assembly;
a pressure sensor in fluid communication with compressed air disposed within an interior volume that is defined by at least a portion of the airflow assembly and the medical device;
a temperature sensor that senses a temperature of water within a water bath within which the medical device is submerged; and
control circuitry that determines a pass or fail result of the leak test on the medical device based on sensor data received from the pressure sensor and the temperature sensed by the temperature sensor.
